(19) United States
(12) Patent Application Publication
(10) Pub. No.: US 2021/0251499 A1
(43) Pub. Date: Aug. 19, 2021

(54) Medical Instrument and Program

(71) Applicant: AMI INC., Kumamoto (JP)

(72) Inventor: Shinpei OGAWA, Kumamoto (JP)

(21) Appl. No.: 15/734,984

(22) PCT Filed: Mar. 20, 2019
(86) PCT No.: PCT/JP2019/011800

§ 371 Date: Dec. 3, 2020

(30) Foreign Application Priority Data
Nov. 15, 2018 (JP) 2018-214405

(51) Int. Cl. A61B 5/0225; A61B 5/00; A61B 5/347

(52) U.S. Cl. CPC A61B 5/0225; A61B 5/347; A61B 5/7264

(57) ABSTRACT

To provide a blood pressure estimating device whereby information relating to the blood pressure of a subject can be acquired by a simple method. A blood pressure estimating device 10 is provided with a frequency analysis unit 11 for specifying the peak value of the heart sound frequency of a subject, and a blood pressure estimating unit 17 for determining information relating to the blood pressure of the subject on the basis of the peak value of the frequency. It is clear that there is a positive correlation between the peak value of the heart sound frequency and the blood pressure value; therefore, by analyzing the heart sound of a subject and specifying a frequency peak value, the blood pressure value of the subject can be easily and accurately estimated.

### TECHNICAL FIELD

The present invention relates to a medical device and a program to acquire blood pressure information of a subject. Specifically, the present invention relates to a device and a program to estimate a blood pressure fluctuation and a blood pressure value from a heart sound of a subject.

### BACKGROUND ART

A device that does not directly measure a blood pressure of a subject with a sphygmomanometer but can estimate the blood pressure of the subject from another biological signal has been conventionally proposed. For example, Patent Document 1 discloses a blood pressure estimating device that estimates a blood pressure value of a subject using a pulse wave signal and an electrocardiogram signal. Patent Document 2 discloses a blood pressure estimating device that estimates a blood pressure value of a subject based on a correlation between a frequency and an amplitude of a heartbeat signal and a blood pressure value.
Patent Document 1: JP-A-2017-176740
Patent Document 2: JP-A-2014-230671

### DISCLOSURE OF THE INVENTION

### Problems to be Solved by the Invention

Nowadays, needs for telemedicine in which a doctor or another person engaged in medical treatment (hereinafter collectively referred to as “doctor”) provides a medical service to a patient who resides in a remote location in real-time have been increasing. In the telemedicine, in addition to a medical examination by interview between the doctor and the patient with a TV phone, in many cases, the doctor preliminarily passes a medical device (such as a digital stethoscope and a sphygmomanometer) to acquire biological information to the patient, and the patient himself/herself transmits the biological information acquired through an operation of the medical device to a terminal of the doctor via the Internet. In this case, it is important that the medical device can be easily used by the patient.

However, like the blood pressure estimating device described in Cited Document 1, when the electrocardiogram signal and the pulse wave signal of the patient are acquired to estimate the blood pressure value, a device, such as a pulse wave sensor and an electrode for electrocardiogram measurement, is required. This causes problems of a complicated configuration of the entire device and an increase in labor for equipping various devices. Similarly to the blood pressure estimating device described in Cited Document 2, since a microwave sensor to measure the frequency and the amplitude of the heartbeat signal is required, a complicated device configuration and a complicated work in measurement are inevitable. Especially, the above-described telemedicine requires the operation of various devices provided with the blood pressure estimating device by the patient himself/herself, but imposing an equipping work and a measurement operation of the complicated medical device, such as the pulse wave sensor, the electrode for electrocardiogram measurement, and the microwave sensor, on the patient is difficult.

Therefore, the main object of the present invention is to provide a medical device that can acquire information on a blood pressure of a subject in a simpler method.

Further, values differ depending on a way of measurement, which is a limit of the existing general non-invasive blood pressure measuring device. For example, for a wrist type, a wrist or for an upper arm type, an upper arm around which a cuff is wound needs to be positioned at a height of the heart. Specifically, when the measurement is performed at a position 10 cm higher than the heart, the value is displayed low by about 8 mmHg, or conversely, when the measurement is performed at a position 10 cm lower than the heart, the value is displayed high by about 8 mmHg. Although the height is adjusted using a towel, the measurement at the height completely identical to the heart is especially difficult for a person not engaged in medical treatment. Therefore, another object of the present invention is to acquire further accurate blood pressure data by directly obtaining data from a heart. That is, one of the objects is to provide a medical device that can acquire information on a blood pressure of a subject more accurately, not only becoming further simple.

### Solutions to the Problems

Through detailed analysis of a heart sound of a subject, the inventor of the present invention has discovered that a peak value of a heart sound frequency and a blood pressure value have a correlation (specifically, a positive correlation) and has found that a blood pressure fluctuation and the blood pressure value can be estimated based on the peak value of the heart sound frequency. The inventor has conceived that acquiring the heart sound and analyzing the frequency easily allows acquiring information on the blood pressure of the subject, thus having completed the present invention. Specifically, the present invention has the following configuration.

A first aspect of the present invention relates to a medical device to obtain blood pressure information of a subject. The medical device according to the present invention includes a frequency analysis unit and a blood pressure estimating unit. The frequency analysis unit identifies a peak value of a heart sound frequency of the subject. For example, it is only necessary to acquire a heart sound data of the subject with a microphone (digital stethoscope) and analyze a frequency of the heart sound data in the frequency analysis unit. Next, the blood pressure estimating unit obtains information on a blood pressure of the subject based on the peak value of the frequency. Specifically, the blood pressure estimating unit performs a predetermined arithmetic operation using the peak value [Hz] of the heart sound frequency as one parameter to estimate a change in blood pressure (blood pressure fluctuation) and a blood pressure value of the subject. Note that the peak value of the heart sound frequency is a peak value of an envelope expressed by a frequency graph. Additionally, the frequency analysis unit may identify the frequency peak value for each beat, the maximum or the minimum frequency peak value among a plurality of beats generated in a certain period, or an average value of the frequency peak value of the plurality of beats generated in the certain period. Further, among heart sounds, the frequency analysis unit may identify a frequency peak value of the first heart sound, a frequency peak value of the second heart sound, or a peak value of a systolic murmur or a diastolic murmur.

FIG. 2 and FIG. 3 illustrate data of a heart sound of an identical person obtained by the actual experiment. The data (1) illustrated in FIG. 2 are the heart sound frequency with the blood pressure of 140/86 mmHg, and the data (2) are the heart sound frequency with the blood pressure of 120/70 mmHg. Both data indicate a heart sound of two beats, and each one beat includes the first heart sound and the second heart sound. It is found that both of the first heart sound and the second heart sound have a positive correlation with a blood pressure fluctuation. Especially, a change in association with the blood pressure fluctuation is larger in the second heart sound. Comparing the peak values of the frequencies of the second heart sounds, the peak value of the frequency of the second heart sound is 150 Hz in the data (1) and the peak value of the frequency of the second heart sound is 130 Hz in the data (2). That is, the higher the peak value of the frequency of the second heart sound is, the blood pressure is high in both of a systole and a diastole. The peak value of the frequency of the first heart sound similarly varies. Since the frequency analysis unit can identify the frequency peak value of the first heart sound as well as the frequency peak value of the second heart sound, it is possible to estimate the fluctuation of the blood pressure from the data of the second heart sound only, the data of the first sound heart only, or the data of the second heart sound and the first heart sound.

The blood pressure information can be obtained through the analysis of the heart sound of the subject like the configuration, and therefore, the blood pressure fluctuation and an estimated value of the blood pressure of the subject can be obtained from the heart sound data acquired with, for example, a microphone (digital stethoscope). As a result, the configuration of the medical device (blood pressure estimating device) can be simplified. Additionally, the heart sound can be easily acquired only by placing the microphone over a chest, thereby ensuring performing the acquisition operation of the heart sound data by the subject himself/herself, without the need of a doctor. The inspection time is a problem of the existing blood pressure measuring device. Since a general non-invasive arterial pressure measurement method needs to press the cuff, one-time blood pressure measurement requires tens of seconds. Meanwhile, invasive arterial pressure measurement allows continuous monitoring in real-time, but the measurement originally requires an artery approach and therefore it takes some time to start the measurement. The present invention starts the measurement at a point when the microphone is placed over a chest, thereby ensuring non-invasive estimation of the blood pressure value in real-time.

With the medical device according to the present invention, the blood pressure estimating unit may obtain the change in the blood pressure of the subject based on changes over time of the peak value of the frequency. As described above, there is a trend that the higher the peak value of the heart sound frequency is, the higher the blood pressure value becomes, so catching the changes over time of the peak value of the heart sound frequency allows obtaining the change in the blood pressure of the subject.

With the medical device according to the present invention, the blood pressure estimating unit may obtain a current estimated value of the blood pressure of the subject based on a data set and a current peak value of the heart sound frequency of the subject. The data set includes a peak value of the heart sound frequency and a previous measured value of the blood pressure of the subject. As long as the data set in which the peak value of the heart sound frequency is made to correspond to the actual measured value of the blood pressure of the subject is preliminarily prepared, only the measurement of the peak value of the heart sound frequency of the subject allows estimating the current blood pressure of the subject.

With the medical device according to the present invention, the blood pressure estimating unit may obtain a current estimated value of the blood pressure of the subject based on a data set and a current peak value of the frequency of the heart sound and an actual measured current value of the heart rate of the subject. The data set includes a peak value of the frequency of the heart sound, an actual measured value of the blood pressure, and an actual measured value of a heart rate of the subject measured in the past. Since the heart rate affects the blood pressure, performing a predetermined arithmetic operation using the heart rate as a parameter together with the peak value of the frequency of the subject and estimating the blood pressure value allow enhancing the accuracy. Note that the heart rate can be calculated from the heart sound data acquired with the microphone or may be measured using an electrocardiograph or the like, separately from the microphone.

With the medical device according to the present invention, the blood pressure estimating unit may obtain a current estimated value of the blood pressure of the subject based on a current peak value of the heart sound frequency of the subject using a learned model obtained by machine learning from a data set of peak values of heart sound frequencies and actual measured values of blood pressures of a plurality of subjects. As long as the data sets of the peak values of the heart sound frequencies and the actually measured values of the blood pressures of multiple subjects are accumulated, the blood pressure is easily estimated from the peak power of the heart sound frequency based on a relation of both data.

The medical device according to the present invention may further include a cardiac function diagnostic unit. The cardiac function diagnostic unit identifies a change in the cardiac function of the subject based on an estimated value of the blood pressure of the subject and an actual measured current value of the blood pressure of the subject. As long as the subject is identical and the cardiac function is identical, the actual measured value and the estimated value of the blood pressures are identical, and therefore presence of some sort of change in the cardiac function is estimated when the difference between them becomes larger than a certain amount. For example, when the difference between the estimated blood pressure value and the actual measured blood pressure value is large, a sign of a cardiac dysfunction, such as a myocardial infarction, a heart failure, and an arrhythmia, is predicted. Therefore, warning notification to the doctor and the subject in such a case allows early detection of these cardiac dysfunctions.

The medical device according to the present invention may further include a cardiac function diagnostic unit that identifies a change in a severity of the valvular heart disease of the subject from the difference between a peak value of a frequency of a systolic murmur or a diastolic murmur of a heart measured in the past and a current peak value of the frequency of the systolic murmur or the diastolic murmur of the heart of the subject. Here, “a peak value of a frequency of a systolic murmur or a diastolic murmur of a heart measured in the past” includes the peak value of the frequency measured in the past of a subject different from the current subject, in addition to this peak value of the frequency measured in the past of the subject identical to the current subject. That is, the current and past frequency peak values of the identical subject may be compared, or the current frequency peak value of a certain subject may be compared with a past frequency peak value of another subject. For example, when data of a peak value of a frequency of a systolic murmur or a diastolic murmur of a heart of a person who is affected with a valvular heart disease is present, a comparison of this data with this frequency peak value of the certain subject allows identifying whether the subject is affected with the valvular heart disease and a change in a severity.

Further, the present invention allows estimating a flow rate and a pressure gradient inside the heart, in addition to the estimation of the blood pressure. FIG. 4 illustrates data of an identical case of aortic valve stenosis. An ejection murmur with a peak value of around 300 Hz is present between the first heart sound and the second heart sound (systole). Seeing data of heart ultrasonography described in the middle stage, it is seen that an aortic valve passing highest flow velocity (AoV Vel) and an aortic valve pressure gradient (AoV PG) change. Peripheries of 300 Hz are enlarged in the drawings on the lower stage, and it is seen that the change in the flow rate or the pressure gradient and the change in the peak value of the sound in the systole have a positive correlation. Since time required for examination substantially shortens and the examination method is simple compared with those in heart ultrasonography, it is shown that the present invention is effective in screening of the change in a severity.

A second aspect of the present invention is a program for the computer. A program according to the present invention causes a computer to execute: a step of identifying a peak value of a heart sound frequency of the subject; and a step of obtaining information on a blood pressure of the subject based on the peak value of the frequency. The program of the present invention may be stored in a recording medium, such as a CD-ROM, or may be downloadable through the Internet.

### Advantageous Effects of the Invention

With the present invention, the simple method, which is the identification of the peak value of the heart sound frequency, allows acquiring the information on the blood pressure of the subject.

### BRIEF DESCRIPTION OF THE DRAWINGS

FIG. 1 is a function block diagram illustrating an example of a configuration of an entire system including a blood pressure estimating device and its peripherals.

FIG. 2 illustrates an example of spectrograms showing a heart sound frequency.

FIG. 3 illustrates an example of spectrograms showing the heart sound frequency.

FIG. 4 illustrates an example of spectrograms showing the heart sound frequency.

### DESCRIPTION OF PREFERRED EMBODIMENTS

Hereinafter, configurations to embody the present invention will be described using the drawings. The present invention is not limited to the configurations described below but includes ones appropriately changed in an obvious range from the following configurations by those skilled in the art.

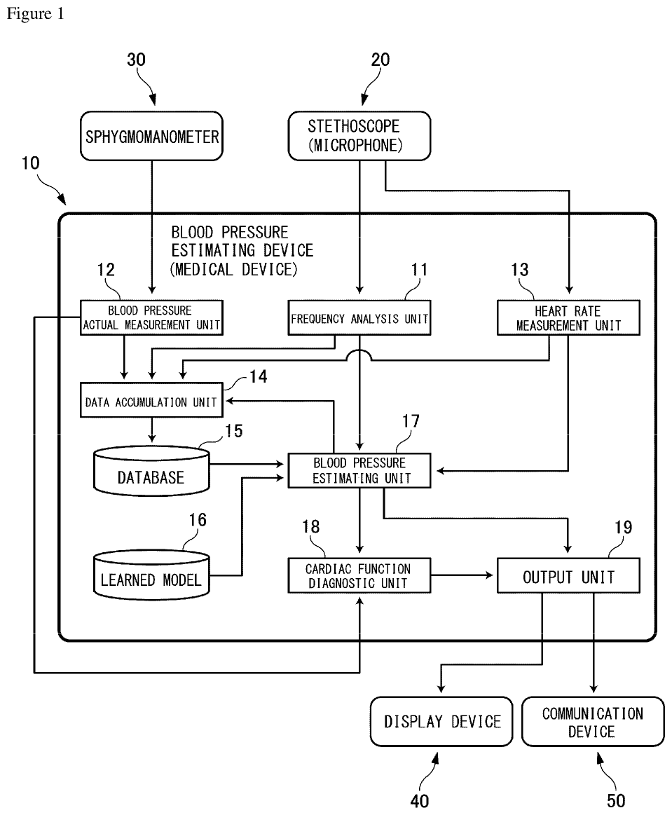

FIG. 1 illustrates the configuration of the entire system including a blood pressure estimating device 10 (medical device), a digital stethoscope 20, a sphygmomanometer 30, a display device 40, and a communication device 50 as its peripherals according to the present invention. The blood pressure estimating device 10 can be achieved by a computer storing specific programs. For example, the blood pressure estimating device 10 may be a mobile terminal, such as a laptop computer, a tablet computer, and a smart phone, or may be a stationary terminal, such as a desktop computer and a web server. When predetermined data is input from the digital stethoscope 20 and the sphygmomanometer 30, the blood pressure estimating device 10 performs an arithmetic operation in accordance with the program and outputs the result of the arithmetic operation to the display device 40 and the communication device 50.

FIG. 1 also illustrates a function block of the blood pressure estimating device 10 achieved by the program specific to the present invention. As illustrated in FIG. 1, the blood pressure estimating device 10 includes a frequency analysis unit 11, a blood pressure actual measurement unit 12, a heart rate measurement unit 13, a data accumulation unit 14, a database 15, a learned model 16, a blood pressure estimating unit 17, a cardiac function diagnostic unit 18, and an output unit 19. That is, this program is described such that the computer achieves these functions.

The frequency analysis unit 11 analyzes the frequency of the heart sound data of the subject obtained from the digital stethoscope 20. The publicly known digital stethoscopes can be used as the digital stethoscope 20. The digital stethoscope 20 internally includes, for example, a microphone for biological sound and directly contacts a skin of the subject to acquire the biological sound (mainly the heart sound). Although a dynamic microphone and a condenser microphone can be used as the microphone for biological sound, to accurately collect a biological sound in a further lower frequency band, the use of a piezoelectric microphone is especially preferred. The piezoelectric microphone converts a sound vibration added to a piezoelectric element into a voltage and basically includes the piezoelectric element and a plurality of electrodes between which the piezoelectric element is sandwiched. The microphone for biological sound only needs to have performance that can collect a frequency (10 Hz to 500 Hz) of the heart sound.

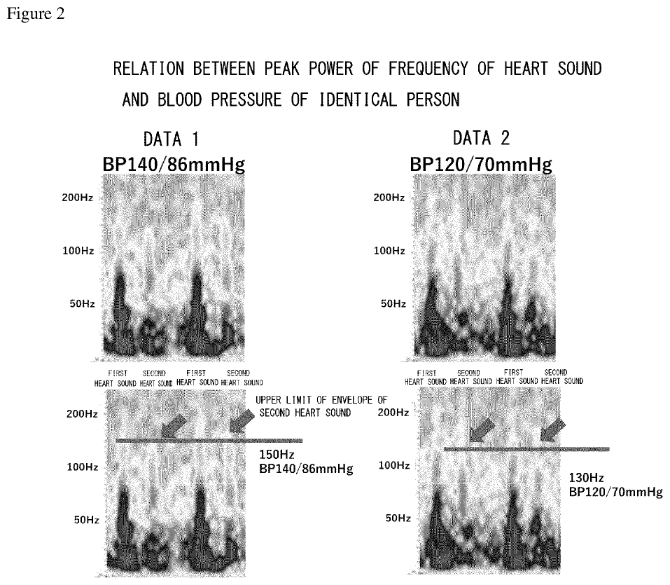

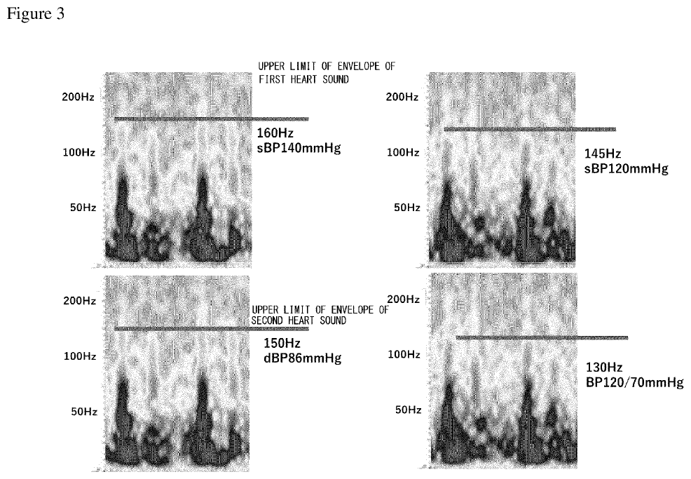

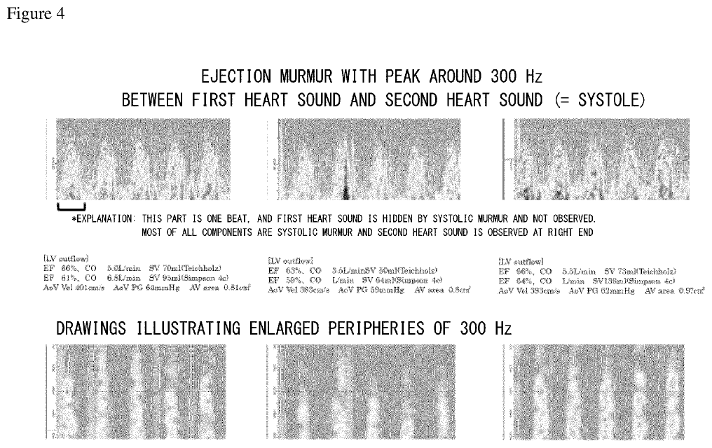

The frequency analysis unit 11 analyzes the heart sound data obtained from the digital stethoscope 20 and identifies the peak value of the frequency [Hz]. The peak value of the frequency is a peak value of an envelope. The frequency analysis unit 11 preferably generates a spectrogram (three-dimensional graph) expressing changes over time of the sound volume of each frequency from the heart sound data (see FIG. 2 to FIG. 4). For example, the spectrogram plots the frequency on a vertical axis and plots the time on a horizontal axis, and expresses the sound volume by a color tone or brightness in the graph (the vertical axis and the horizontal axis can be switched). Note that FIG. 2 to FIG. 4 are illustrated in monochrome, but a frequency bandwidth in which the sound volume is high is expressed by red and the frequency bandwidth in which the sound volume is low is expressed by blue actually. The spectrogram allows easily identifying the peak value of the heart sound frequency.

The heart sound is a sound generated in association with a pulse of a heart and generates the first heart sound and the second heart sound. Among these sounds, a sound generated immediately after the start of the systole of the heart is the first heart sound and a sound generated at the border between the systole and the diastole is the second heart sound. The heart sound includes heart murmurs in some cases. The heart murmur is a sound generated in association with the heartbeat but is not generated in the normal heart. The frequency analysis unit 11 preferably identifies the frequency peak value of the sound of both or any one of the first heart sound component or a second component heart sound among these sounds included in the heart sound. As described later, the estimated value of a systolic blood pressure can be obtained from the frequency peak value of the first heart sound component and the estimated value of a diastolic blood pressure can be obtained from the frequency peak value of the second heart sound component. Additionally, when the heart murmur is included in the heart sound, the frequency analysis unit 11 may identify a frequency peak value of a heart murmur component.

The blood pressure actual measurement unit 12 measures the actual blood pressure of the subject from the data obtained from the sphygmomanometer 30. The publicly known sphygmomanometer 30 can be used. The sphygmomanometer 30, for example, includes a cuff wound around an arm of the subject, a pump that supplies an air to the inside of the cuff, a pressure sensor that converts an air pressure inside the cuff into an electrical signal, and so on. For example, the blood pressure measurement unit 12 measures the actual blood pressure of the subject based on the electrical signal obtained from this pressure sensor. The blood pressure value measured by the blood pressure measurement unit 12 may be a systolic blood pressure, a diastolic blood pressure, or the both. The blood pressure actual measurement unit 12 preferably measures the blood pressure in the heart systole and the blood pressure in the heart diastole. Although a method for measuring the blood pressure is not specifically limited, the known invasive arterial pressure measurement or non-invasive arterial pressure measurement, for example, may be employed. The blood pressure can be measured from the pulse pressure of the subject.

The heart rate measurement unit 13 measures the heart rate of the subject based on the heart sound data obtained from, for example, the digital stethoscope 20. For example, the heart rate can be obtained by counting periodicity of the strength and weakness of the sound components included in the heart sound data for a certain period. While the example illustrated in FIG. 1 obtains the heart rate from the heart sound data acquired by the digital stethoscope 20 to make the entire system compact, an electrocardiograph (such as electrodes) may be separately connected to the blood pressure estimating device 10 to measure the heart rate.

The data accumulation unit 14 causes the database 15 to store data of the peak value of the heart sound frequency identified in the frequency analysis unit 11, the blood pressure value measured in the blood pressure actual measurement unit 12, and the heart rate measured in the heart rate measurement unit 13. Especially, in the initial examination, the blood pressure and the heart sound of the subject are acquired simultaneously or under identical conditions. The actually measured blood pressure values obtained from the blood pressure and the heart sound, the heart sound frequency peak value, and the heart rate are preferably associated and stored in the database as one data set. A plurality of data sets may be generated for one subject. Even when the data set as described above is not generated, when the peak power of the heart sound frequency, the actual measured value of the blood pressure, and the heart rate are obtained, the data accumulation unit 14 preferably stores these values in the database 15 as needed. Further, as described later, the blood pressure estimating unit 17 calculates the estimated value of the blood pressure of the subject based on the peak value of the heart sound frequency. The data accumulation unit 14 preferably associates this estimated value of the blood pressure with the source peak value of the heart sound frequency and stores it in the database 15. Thus, the data accumulation unit 14 appropriately accumulates the various pieces of biological data obtained in the blood pressure estimating device 10 in the database 15.

The learned model 16 is model data in which parameters (what is called “weights”) are adjusted through machine learning on the biological data of multiple subjects. For example, performing the machine learning, such as deep learning, using the data sets of the peak values of the heart sound frequencies and the actually measured values of the blood pressures of the multiple subjects as teaching data generates the learned model 16. In this case, reference to this learned model 16 with the peak value of the heart sound frequency of a certain subject as an input value allows obtaining the estimated value of the blood pressure corresponding to the input value as an output value. The blood pressure estimating device 10 may preliminarily include the learned model 16. Note that this learned model 16 is not an indispensable element.

The blood pressure estimating unit 17 estimates the blood pressure fluctuation and the blood pressure value of the subject based on at least the peak value of the heart sound frequency obtained from the frequency analysis unit 11. The blood pressure estimating unit 17 can perform a predetermined arithmetic operation according to a situation, such as a mode that obtains only the blood pressure fluctuation of the subject and a mode that obtains the estimated value of the blood pressure of the subject. Details of the arithmetic operation mode by the blood pressure estimating unit 17 will be described later.

The cardiac function diagnostic unit 18, for example, compares the estimated value of the blood pressure obtained by the blood pressure estimating unit 17 with the actual measured value of the blood pressure measured in the blood pressure actual measurement unit 12, and determines whether the cardiac function of the subject is normal, and the severity of the cardiac dysfunction or its change based on the difference and the ratio of these values. For example, the actual measured value and the estimated value of the blood pressure of the subject are obtained simultaneously or under the identical conditions using the digital stethoscope 20 and the sphygmomanometer 30. When the difference and the ratio of these values are equal to or more than certain thresholds, the cardiac function diagnostic unit 18 determines that the cardiac function is abnormal. According to the difference between the actual measured value and the estimated value of the blood pressure, presence of a sign of the cardiac dysfunction, such as myocardial infarction, heart failure, and arrhythmia, may be identified.

Additionally, the cardiac function diagnostic unit 18 can identify the severity of the valvular heart disease from the difference between the peak value of the frequency of the systolic murmur or the diastolic murmur of the heart measured in the past and the current peak value of the frequency of the systolic murmur or the diastolic murmur of the heart of the subject. For example, a comparison between the peak value of a heart sound frequency of a person who has been already affected with the valvular heart disease and the peak value of the heart sound frequency of a certain subject allows determining whether the subject is affected with the valvular heart disease and its severity. Additionally, a comparison of the peak value of the heart sound frequency measured in the past with the current peak value of the heart sound frequency of a certain subject allows identifying the change (the exacerbation or improvement) in the severity of the valvular heart disease.

The output unit 19 outputs the results obtained from the blood pressure estimating unit 17 and the cardiac function diagnostic unit 18 to the display device 40 and the communication device 50. For example, the output unit 19 can display the blood pressure fluctuation, the estimated value of the blood pressure, and the presence/absence of the cardiac dysfunction in the display device 40. The output unit 19 may display an analysis result (frequency peak value) of the heart sound frequency, the actual measured value of the blood pressure, or information on the heart rate in the display device 40. Further, the output unit 19 can transmit various pieces of the information obtained by the blood pressure estimating device 10 to an external terminal via an information communication network, such as the Internet, through the communication device 50. For example, it is preferred that the subject himself/herself operates the blood pressure estimating device 10 and its peripherals 20, 30, 40, 50 to measure the estimated value of the blood pressure and transmits the measured value to a terminal of the doctor residing in a remote location. Thereby, the estimated value of the blood pressure can be used for telemedicine.

Next, an example of the arithmetic operation mode that can be performed by the blood pressure estimating unit 17 will be described.

### [1. Estimation of Blood Pressure Fluctuation]

The blood pressure estimating unit 17 can estimate the blood pressure fluctuation of the subject based on the change over time of the peak value of the heart sound frequency. Since the peak value of the heart sound frequency and the blood pressure value are related to positive correlation, when the peak value of the heart sound frequency changes, it can be estimated that the blood pressure value similarly changes. When the blood pressure varies, the frequency peak value of a second heart sound component makes the biggest change among the heart sounds, and therefore the frequency peak value of the second heart sound component is preferably referred for estimation of the blood pressure fluctuation. Although it is medically known that the second heart sound increases at a high blood pressure in auscultatory findings of a high blood pressure patient (that is, power of the second heart sound increases), the inventor has found new knowledge that the blood pressure and the peak value of the heart sound frequency are related to positive correlation in real-time, and the blood pressure can be estimated from the change in the peak value of the frequency of the second heart sound. Further, for further accurate identification of blood pressure fluctuation, a data set as a reference in which an actually measured normal blood pressure value is associated with the peak value of the heart sound frequency may be preliminarily generated, and the blood pressure estimating unit 17 may compare a peak value of a heart sound frequency measured after that with this data set to obtain the extent of change in the peak value of the heart sound frequency. When the change in the peak value of the heart sound frequency is large, it can be determined that the blood pressure greatly changes by the amount.

### [2. Estimation of Blood Pressure Value Based on Heart Sound Frequency]

The blood pressure estimating unit 17 can obtain the estimated value of the blood pressure (the systolic blood pressure or the diastolic blood pressure) based on the following relational equation using the peak value of the heart sound frequency as a parameter.

$$Estimated blood pressure = ( α \times \frac{D - C}{C} + 1 ) \times A \qquad [ Formula 1 ]$$

α denotes a coefficient related to the peak value of the heart sound frequency used to estimate the blood pressure. To distinguish between the systolic blood pressure and the diastolic blood pressure, different values between the systolic blood pressure and the diastolic blood pressure may be used for α. A denotes a blood pressure value as a reference measured in the past. C denotes a frequency peak value (a value measured simultaneously with or under a condition identical to that of A) of the second heart sound component as a reference measured in the past. D denotes a frequency peak value of the second heart sound component measured this time. Values measured when the subject is in a normal health condition are preferably used for A and C. Any given value can be employed for the coefficient α.

The equation 1 is one example. When the fact that the peak value of the heart sound frequency and the blood pressure value show the positive correlation is applied, by identifying the peak value of the heart sound frequency, the blood pressure value corresponding to it can be estimated.

### [3. Estimation of Blood Pressure Value Based on Heart Sound Frequency and Heart Rate]

The blood pressure estimating unit 17 can obtain the estimated values of the systolic blood pressure and the diastolic blood pressure based on the following relational equation using the peak value of the heart sound frequency and the heart rate as parameters.

$$Estimated blood pressure = ( β \times \frac{F - E}{E} + 1 ) \times ( γ \times \frac{G - H}{G} + 1 ) \times B \qquad [ Formula 2 ]$$

B denotes a blood pressure value as a reference measured in the past. β denotes a coefficient related to the peak value of the heart sound frequency used to estimate the systolic blood pressure. E denotes a frequency peak value (measured simultaneously with or under a condition identical to that of B) of the second heart sound component as a reference measured in the past. F denotes a frequency peak value of the second heart sound component measured this time. γ denotes a coefficient related to a heart rate used to estimate the systolic blood pressure. G denotes a heart rate (measured simultaneously with or under a condition identical to that of B) as a reference measured in the past. H is a heart rate measured this time. Values measured when the subject is in a normal health condition are preferably used for B, E, and G. The coefficients β, γ can employ any values. To distinguish between the systolic blood pressure and the diastolic blood pressure, different values between the systolic blood pressure and the diastolic blood pressure may be used for β and γ.

The equation 2 is one example. When the fact that the peak value of the heart sound frequency and the heart rate show the positive correlation to the blood pressure value is applied, by actually measuring the peak value of the heart sound frequency and the heart rate, the blood pressure value corresponding to it can be estimated. Especially, since it is known that the blood pressure value changes due to the heart rate, the use of both of the peak value of the heart sound frequency and the heart rate as the parameters allows further accurately estimating the blood pressure value.

In the present specification, the embodiments of the present invention have been described above by referring to the drawings to express the contents of the present invention. However, the present invention is not limited to the above-described embodiments and encompasses changed configurations and improved configurations obvious for those skilled in the art based on the matters described in the present specification.

### DESCRIPTION OF REFERENCE SIGNS

10 . . . blood pressure estimating device (medical device)
11 . . . frequency analysis unit
12 . . . blood pressure actual measurement unit
13 . . . heart rate measurement unit
14 . . . data accumulation unit
15 . . . database
16 . . . learned model
17 . . . blood pressure estimating unit
18 . . . cardiac function diagnostic unit
19 . . . output unit
20 . . . digital stethoscope
30 . . . sphygmomanometer
40 . . . display device
50 . . . communication device

1. A medical device comprising:
a frequency analysis unit that identifies a peak value of a frequency of a heart sound of a subject; and
a blood pressure estimating unit that obtains the change or the estimated value of a blood pressure of the subject based on the peak value of the frequency.

2. The medical device according to claim 1, wherein
the blood pressure estimating unit obtains the change in the blood pressure of the subject based on changes over time of the peak value of the frequency.

3. The medical device according to claim 1, wherein
the blood pressure estimating unit obtains a current estimated value of the blood pressure of the subject based on a data set and a current peak value of the frequency of the heart sound of the subject, the data set including a peak value of the frequency of the heart sound and an actual measured value of the blood pressure of the subject measured in a past.

4. The medical device according to claim 1, wherein
the blood pressure estimating unit obtains a current estimated value of the blood pressure of the subject based on a data set and a current peak value of the frequency of the heart sound and an actual measured current value of the heart rate of the subject, the data set including a peak value of the frequency of the heart sound, an actual measured value of the blood pressure, and an actual measured value of a heart rate of the subject measured in the past.

5. The medical device according to claim 1, wherein
the blood pressure estimating unit obtains a current estimated value of the blood pressure of the subject based on a current peak value of the frequency of the heart sound of the subject using a learned model obtained by machine learning from a data set of peak values of heart sound frequencies and blood pressures of a plurality of subjects.

6. The medical device according to claim 3 or claim 4, further comprising
a cardiac function diagnostic unit that identifies a change in a cardiac function of the subject based on an estimated value of the blood pressure of the subject and an actual measured current value of the blood pressure of the subject.

7. The medical device according to claim 1, further comprising
a cardiac function diagnostic unit that identifies a change in a severity of a valvular heart disease of the subject from the difference between a peak value of a frequency of a systolic murmur or a diastolic murmur of a heart measured in a past and a current peak value of the frequency of the systolic murmur or the diastolic murmur of the heart of the subject.

8. A program for causing a computer to execute:
a step of identifying a peak value of a heart sound frequency of a subject; and
a step of obtaining a change or an estimated value of a blood pressure of the subject based on the peak value of the frequency.
